(19) United States
(12) Patent Application Publication
(10) Pub. No.: US 2023/0096761 A1
(43) Pub. Date: Mar. 30, 2023

(54) NOVEL COMPOSITION BASED ON POLYCAFFEOYLQUINIC ACIDS, COSMETIC USE THEREOF AND COSMETIC COMPOSITIONS COMPRISING SAME

(71) Applicant: Societe D'Exploitation De Produits Pour Les Industries Chimiques Seppic, Paris Cedex 07 (FR)

(72) Inventors: Catherine KERN, Paris Cedex 07 (FR); Christine GARCIA, Castres (FR); Christian GOMBERT, Paris Cedex 07 (FR); Philippe MSIKA, Paris Cedex 07 (FR)

(21) Appl. No.: 18/059,108

(22) Filed: Nov. 28, 2022

Related U.S. Application Data
(62) Division of application No. 17/046,636, filed on Oct. 9, 2020.

(30) Foreign Application Priority Data
Apr. 12, 2018 (FR) 1853224

(51) Int. Cl. A61K 8/37; A61K 8/9789; A61P 17/16; A61K 31/216; A61K 36/28; A61Q 19/00

(52) U.S. Cl. CPC A61K 8/375; A61K 8/9789; A61P 17/16; A61K 31/216; A61K 36/28; A61Q 19/007; A61K 9/0014

(57) ABSTRACT

Disclosed is a composition including, per 100% of its mass: a) 60.0% to 75.0% by mass of an organic solvent (OS₁) chosen from 1,2-propanediol, 1,3-propanediol, 1,4-butanediol, 1,3-butanediol, 1,2-butanediol, 2-methyl-2,4-pentanediol, 1,6-hexanediol, 1,8-octanediol, or a mixture of these compounds; b) 0.1% to 2.0% by mass of a composition (ES) including an amount by mass x₁, expressed as mass equivalent of 1-O-(2-caffeoyl)maloyl-3,5-di-O-catfeoylquinic acid, of greater than or equal to 200 mg/g of a compound of general formula (I):

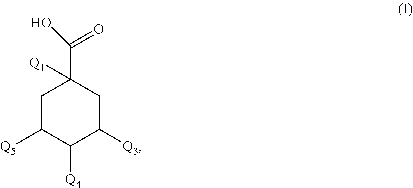

in which Q₁, Q₂, Q₃, Q₄ and Q₅ represent, independently of each other, a hydroxyl radical or a salt thereof or a radical chosen from caffeoyl, maloyl, caffeoyl maloyl and maloyl caffeoyl radicals, it being understood that at least one of these radicals Q₁, Q₂, Q₃, Q₄ and Q₅ represents neither the —OH radical nor a salt thereof; and c) 20.0% to 35.0% by mass of water. Related treatments are also disclosed.

This application is a divisional of U.S. application Ser. No. 17/046,636 filed Oct. 9, 2020, which is the U.S. national phase of International Application No. PCT/FR2019/050840 filed Apr. 10, 2019 which designated the U.S. and claims priority to French Patent Application No. 1853224 filed Apr. 12, 2018, the entire contents of each of which are hereby incorporated by reference.

### BACKGROUND OF THE INVENTION

### Field of the Invention

The present invention relates to a novel composition comprising polysubstituted quinic acid derivatives, and more particularly to an extract of the plant Arctium lappa comprising said derivatives, to the process for preparing same, and to the use of the novel composition comprising polysubstituted quinic acid derivatives for preparing formulations for topical use intended for moisturizing the skin, and more particularly the epidermis of human or animal skin.

The invention mainly finds an application in the cosmetic and dermopharmaceutical fields, but also in the field of the textile industry, for example for treating woven or knitted synthetic or natural textile fibers, or in the field of the paper industry, for example for manufacturing paper for sanitary or domestic use.

### Description of the Related Art

The skin is an atypical organ of the human body, extremely thin with regard to its extent but also the heaviest organ of an individual. One of the characteristics of the skin lies in the fact that it is an interface organ, a boundary organ, between the internal medium (human body) and the external environment. For this reason, and with the flora which covers it and inhabits it, the skin is the first protective barrier of the human body.

Due to its interface position with the external environment, the skin is subjected to numerous daily stresses, for instance contact with clothing, changes in temperature, changes in humidity levels, changes in pressure, or even to attacking factors, for instance contact with certain chemicals which have or may have a very acidic, or very basic, or irritant nature, with chemicals regarded as polluting agents.

The skin is composed of layers of different tissues:
the epidermis, composed of keratinocytes, is its outermost part, followed by
the dermis, which is a connective tissue mainly composed of fibroblasts and of extracellular matrix, and
the hypodermis, consisting of adipocytes, which is the deepest part and the part furthest from the external environment.

The skin performs various functions in the interest of the entire system which it shelters, among which are included the following:
a mechanical barrier function in order to guarantee the integrity of the internal medium of the body,
an emunctorial function directed toward secreting sweat based on water, on salts and on acidic waste,
a function of regulating the temperature of the body, and contains many other regulatory mechanisms, for instance its mechanism of adaptation and of protection against ultraviolet radiation (adaptive pigment coloring by the production of melanin), for instance a system for immune monitoring by the presence of macrophages or dendritic cells.

Human skin also constitutes the first image seen by others.

Consequently, improving its appearance is a subject of constant concern for humans. The skin is the reflection of a state of well-being, often associated with youthfulness, and, conversely, with a state of fatigue and/or aging. As a result, preserving and improving the state of the outermost layer of the skin, namely the epidermis, is a major focus for the research conducted by the cosmetics industries.

At the periphery of the epidermis is an upper cornified layer known as the stratum corneum, which is the first layer of the epidermis to suffer the stresses of external origin, such as variations in external climatic conditions (temperature, pressure, hygrometry) or mechanical stresses.

The stratum corneum is more particularly in contact with the skin microbiota.

For the purposes of the present patent application, the term “skin microbiota” denotes a population of specialized or opportunistic microorganisms, for instance bacteria, fungi, yeasts, and the like, which live on the surface of the skin.

The skin microbiota cannot be defined in a specific and generalized manner for all individuals. Since the launch in 2007 of the National Institute of Health's “Human Microbiome Project” (HMP), researchers have been able to observe large topographical variations in the human microbiota and also large differences between individuals.

At least nineteen phyla have been identified, the four main ones of which are Actinobacteria (51.8%), Firmicutes (24.4%), Proteobacteria (16.5%) and Bacteroidetes (6.3%). The genera predominantly identified are Corynebacterium, Propionibacterium and Staphylococcus. The abundance of each group is strongly dependent on the different locations. The fungal organisms isolated from the skin are of the genus Malassezia spp. Furthermore, mites of the genus Demodex are also present and reside in the pilosebaceous units, most often of the surface of the face.

This microbiota feeds both on molecules excreted by the skin (lipids, proteins, etc.) and on compounds secreted by communities of microorganisms, demonstrating real interaction within this microbiota. In addition, this relationship with the host constitutes a true symbiosis.

Bacteria can be commensal when they live in contact with the cutaneo-mucous covering of a host without causing damage. A balance is then established between the individual and the various commensal flora of the skin and mucous membranes, but this balance is constantly threatened by the physical or chemical attacks undergone by the stratum corneum, for instance pollution, variations in temperature, ultraviolet radiation, the intensive use of detergent surfactants, stress, etc. Alongside these commensal bacteria are unwanted and/or pathogenic transient opportunistic bacteria.

Staphylococcus epidermidis (S. epidermidis) constitutes more than 90% of the resident flora under aerobic conditions present in the stratum corneum. The resident flora is also composed of anaerobic bacteria belonging to the division of the Actinobacteria, such as Propionibacterium acnes (P. acnes), frequently found in sebaceous regions, for instance the back, the face and the scalp.

Whereas the normal skin flora constitutes a defence for the host, an increase or reduction in the bacterial composition (dysbiosis) may lead to a physiological imbalance, for instance by slowing the differentiation of the keratinocytes contained in the epidermis, which leads to weakening of the skin's barrier function and, consequently, renders the stratum corneum more vulnerable to dehydration and to dryness.

This is why improving the appearance or maintaining the satisfactory appearance of human skin consists notably in maintaining or reinforcing the barrier function of the epidermis so as to maintain or achieve a state of moisturization of the stratum corneum at an optimum and satisfactory level. This also makes it possible to avoid esthetic and physiological drawbacks associated with dryness of the skin.

Many solutions have already been provided to solve the problems of skin dryness caused by dehydration of the stratum corneum, notably by developing moisturizing compositions.

An improvement in or preservation of the moisturization of the skin, and more particularly of the stratum corneum, may be imagined by placing the skin in contact with compounds which have a “moisturizing effect”.

The term “moisturizing effect” means an increase in the degree of moisturization of the stratum corneum resulting from the topical application of a chemical substance or of a chemical composition onto the surface of the skin to be treated.

The following are moisturizers for the skin, and more particularly for very dry and destructured skin:
occlusive agents, which are characterized by their ability to form an impermeable film on the surface of the skin and thus to greatly reduce the evaporation of water at the surface of the epidermis. Examples of such agents include mineral oils, for instance petroleum jelly or liquid paraffins, glycerol, shea butter (Butyrospermum parkii butter), beeswax (Cera alba), certain plant oils such as wheat germ oil, coconut oil, cocoa butter, lanolin and silicone derivatives;
emollients, which are characterized by their ability to fill the intercellular spaces existing between corneocytes (cells of the cornified layer of the epidermis); they also limit the evaporation of water from the epidermis, but to a lesser extent than occlusive agents. Examples of such agents include ceramides, linoleic acid and certain plant oils such as sweet almond oil or jojoba oil;
film-forming agents, which are characterized by their ability to associate with water to form semipermeable hydrogels; they thus participate in modulating the evaporation of water from the stratum corneum. Examples of such agents include collagen, elastin, DNA, pectin, gelatin, chitosan, or glycosaminoglycans such as hyaluronic acid;
humectants, which are hydrophilic substances characterized by their high hygroscopic power, i.e. their ability to retain water. They thus contribute toward enabling the stratum corneum to conserve both the water it contains and the water provided by the cosmetic formula. Examples of such agents include urea, glycerol, lactic acid, amino acids, sodium lactate, propylene glycol, polyethylene glycols, α-hydroxy acids or sorbitol. Glycerol, urea and lactic acid are the humectants most frequently used in moisturizing cosmetic compositions, most particularly glycerol for its very competitive price.

However, certain humectants such as glycerol also have an immediate occlusive effect, which is not desired for normal skin whose barrier function is not deficient.

Furthermore, some of them, such as glycerol, cause certain skin and mucous membrane irritations in the case of particularly sensitive people.

Among the plant extracts that may be used for their actions on human microbiota, mention may be made of a lyophilized extract of burdock (or Arctium lappa) leaves for which antibacterial activity has been demonstrated and more particularly activity against oral microorganisms, appearing more effective against bacteria associated with endodontic pathogens, such as Bacillus subtilis. Candida albicans, Lactobacillus acidophilus and Pseudomonas aeruginosa (1).

The Chinese patent application published under the number CN 104304955 A discloses a complex composition obtained by mixing various plant parts, among which are burdock root, followed by centrifugation to obtain the juice of this complex mixture of plant parts. It also discloses that the medicinal herbs used, such as burdock root, are used in the long term for their beneficial effects on the kidneys, on moisturization of the skin and for retarding aging.

The Chinese patent application published under the number CN 105232438 A discloses a mask for moisturizing the skin, comprising a complex mixture in which are many root extracts, among which is an extract of burdock root.

The Chinese patent application published under the number CN 107157800 A discloses a complex formulation comprising polysaccharides derived from burdock root, with moisturizing, antibacterial, antiinflammatory, antiaging and anti-fatigue effects.

The Chinese patent application published under number CN106074663 A discloses a composition of plant extracts comprising an extract of Milo wood, an extract of burdock root and an extract of honeysuckle, and more particularly describes that the extract of burdock root treats dry skin, acute pruritus, inflammation, scars and other symptoms, by inhibiting inflammatory factors induced by different causes (external stress or genetic factors), improving the immune activity and the antioxidant activity of the skin, soothing and repairing the skin.

### SUMMARY OF THE INVENTION

In the context of their research concerning improving the moisturization of the skin, the inventors focused on developing a novel composition comprising polysubstituted quinic acids (or PSQs) which have positive effects on moisturization of human skin.

According to a first aspect, a subject of the invention is a composition (C₁) comprising, per 100% of its mass:

a)- from 60.0% by mass to 75.0% by mass of an organic solvent (OS₁) chosen from 1,2-propanediol, 1,3-propanediol, 1,4-butanediol, 1,3-butanediol, 1,2-butanediol, 2-methyl-2,4-pentanediol, 1,6-hexanediol, 1,8-octanediol, or a mixture of these compounds;

b)- from 0.1% by mass to 2.0% by mass of a composition (ES) comprising an amount by mass x₁, expressed as mass equivalent of 1-O-(2-caffeoyl)maloyl-3,5-di-O-caffeoylquinic acid, of greater than or equal to 200 mg/g of at least one compound of general formula (I):

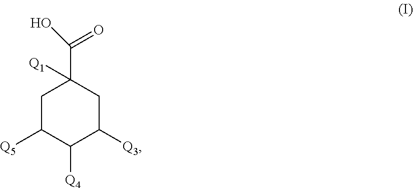

in which Q₁, Q₂, Q₃, Q₄ and Q₅ represent, independently of each other, a hydroxyl radical or a salt thereof or a radical chosen from:

(i)- the caffeoyl radical of formula (II):

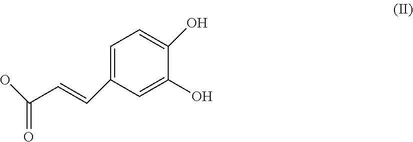

(ii)- the maloyl radical of formula (IIIa) or (IIIb):

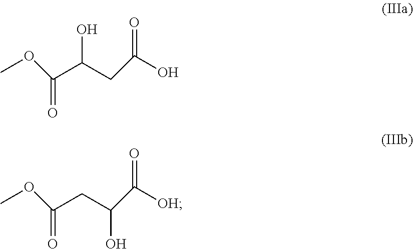

(iii)- the caffeoyl maloyl radical of formula (IVa) or (IVb):

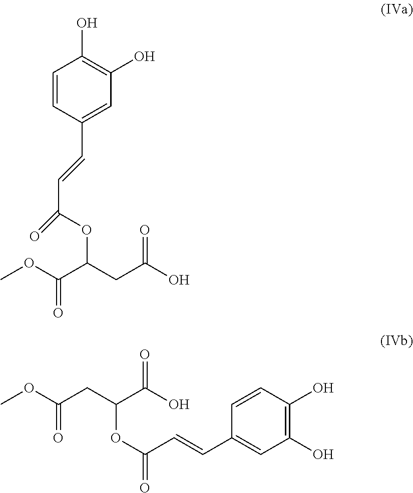

(iv)- the maloyl caffeoyl radical of formula (Va), (Vb), (Vc) or (Vd),

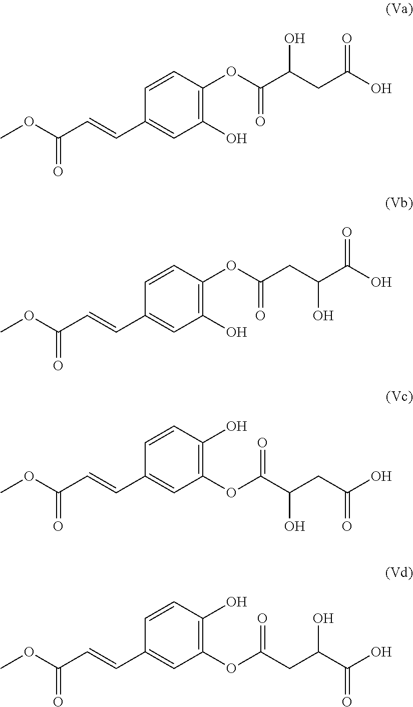

it being understood that at least one of these radicals Q₁, Q₂, Q₃, Q₄ and Q₅ represents neither the —OH radical nor a salt thereof, and

c)- from 20.0% by mass to 35.0% by mass of water.

For the purposes of the present invention, the term “said amount by mass x₁ being expressed as mass equivalent of 1-O-(2-caffeoyl)maloyl-3,5-di-O-caffeoylquinic acid” means that the amount by mass x₁ was determined by performing a quantitative analytical method, such as UHPLC-MS (Ultra High Performance Liquid Chromatography-Mass Spectra), using as reference standard a 1-O-(2-caffeoyl)maloyl-3,5-di-O-catfeoylquinic acid standard previously isolated and purified to a content of greater than or equal to 99%.

Such a quantitative analysis of UHPLC-MS type was performed with apparatus of UHPLC-MS Shimadzu_Nexera_LCMS 2020 type, equipped with a diode array detector and adjusted to a wavelength of 330 nanometers, with a column of Kinetex 2.6u XB-C18 100A, 100×2.1 type, and employing a mobile phase A composed of water and of 0.1% by mass of formic acid and a mobile phase B consisting of acetonitrile.

Among the compounds of general formula (I) present in the composition (ES), mention may be made of:
the compounds of the family of DiCaffeoylQuinic acids (DCQ), as described in table 1 below:

TABLE 1
DiCaffeoylQuinic Acid (DCQ)

| | | | |
|---|---|---|---|
| Q1 | Q3 | Q4 | Q5 |

1,3-Di-O-caffeoylquinic acid (1,3-DCQ)

| | | | |
|---|---|---|---|
| Caffeoyl | Caffeoyl | OH | OH |

1,4-Di-O-caffeoylquinic acid (1,4-DCQ)

| | | | |
|---|---|---|---|
| Caffeoyl | OH | Caffeoyl | OH |

1,5-Di-O-caffeoylquinic acid (1,5-DCQ)

| | | | |
|---|---|---|---|
| Caffeoyl | OH | OH | Caffeoyl |

3,4-Di-O-caffeoylquinic acid (3,4-DCQ)

| | | | |
|---|---|---|---|
| OH | Caffeoyl | Caffeoyl | OH |

3,5-Di-O-caffeoylquinic acid (3,5-DCQ)

| | | | |
|---|---|---|---|
| OH | Caffeoyl | OH | Caffeoyl |

4,5-Di-O-caffeoylquinic acid (4,5-DCQ)

| | | | |
|---|---|---|---|
| OH | OH | Caffeoyl | Caffeoyl |

the compounds of the family of TriCaffeoylQuinic acids (TCQ), as described in table 2 below:

TABLE 2
TriCaffeoylQuinic Acid (TCQ)

| | | | |
|---|---|---|---|
| Q1 | Q3 | Q4 | Q5 |

1,3,4-Tri-O-caffeoylquinic acid (1,3,4-TCQ)

| | | | |
|---|---|---|---|
| Caffeoyl | Caffeoyl | Caffeoyl | OH |

1,3,5-Tri-O-caffeoylquinic acid (1,3,5-TCQ)

| | | | |
|---|---|---|---|
| Caffeoyl | Caffeoyl | OH | Caffeoyl |

1,4,5-Tri-O-caffeoylquinic acid (1,4,5-TCQ)

| | | | |
|---|---|---|---|
| Caffeoyl | OH | Caffeoyl | Caffeoyl |

3,4,5-Tri-O-caffeoylquinic acid (1,3,4-TCQ)

| | | | |
|---|---|---|---|
| OH | Caffeoyl | Caffeoyl | Caffeoyl |

the compounds of the family of MaloylTriCaffeoylQuinic acids (m-TCQ), as described in table 3 below:

TABLE 3
MaloylTriCaffeoylQuinic acid (m-TCQ)

| | | | |
|---|---|---|---|
| Q1 | Q3 | Q4 | Q5 |

1-O-Maloyl-(3,4,5-tri-O-caffeoyl)quinic acid

| | | | |
|---|---|---|---|
| Maloyl | Caffeoyl | Caffeoyl | Caffeoyl |

3-O-Maloyl-(1,4,5-tri-O-caffeoyl)quinic acid

| | | | |
|---|---|---|---|
| Caffeoyl | Maloyl | Caffeoyl | Caffeoyl |

4-O-Maloyl-(1,3,5-tri-O-caffeoyl)quinic acid

| | | | |
|---|---|---|---|
| Caffeoyl | Caffeoyl | Maloyl | Caffeoyl |

5-O-Maloyl-(1,3,4-tri-O-caffeoyl)quinic acid

| | | | |
|---|---|---|---|
| Caffeoyl | Caffeoyl | Caffeoyl | Maloyl |

the compounds of the family of MaloylDiCaffeoylQuinic acids (m-DCQ), as described in table 4 below:

TABLE 4
MaloylDiCaffeoylQuinic Acid (m-DCQ)

| | | | |
|---|---|---|---|
| Q1 | Q3 | Q4 | Q5 |

4-O-Maloyl-(1,3-di-O-caffeoyl)quinic acid

| | | | |
|---|---|---|---|
| Caffeoyl | Caffeoyl | Maloyl | OH |

5-O-Maloyl-(1,3-di-O-caffeoyl)quinic acid

| | | | |
|---|---|---|---|
| Caffeoyl | Caffeoyl | OH | Maloyl |

3-O-Maloyl-(1,4-di-O-caffeoyl)quinic acid

| | | | |
|---|---|---|---|
| Caffeoyl | Maloyl | Caffeoyl | OH |

5-O-Maloyl-(1,4-di-O-caffeoyl)quinic acid

| | | | |
|---|---|---|---|
| Caffeoyl | OH | Caffeoyl | Maloyl |

3-O-Maloyl-(1,5-di-O-caffeoyl)quinic acid

| | | | |
|---|---|---|---|
| Caffeoyl | Maloyl | OH | Caffeoyl |

4-O-Maloyl-(1,5-di-O-caffeoyl)quinic acid

| | | | |
|---|---|---|---|
| Caffeoyl | OH | Maloyl | Caffeoyl |

1-O-Maloyl-(3,4-di-O-caffeoyl)quinic acid

| | | | |
|---|---|---|---|
| Maloyl | Caffeoyl | Caffeoyl | OH |

5-O-Maloyl-(3,4-di-O-caffeoyl)quinic acid

| | | | |
|---|---|---|---|
| OH | Caffeoyl | Caffeoyl | Maloyl |

1-O-Maloyl-(3,5-di-O-caffeoyl)quinic acid

| | | | |
|---|---|---|---|
| Maloyl | Caffeoyl | OH | Caffeoyl |

4-O-Maloyl-(3,5-di-O-caffeoyl)quinic acid

| | | | |
|---|---|---|---|
| OH | Caffeoyl | Maloyl | Caffeoyl |

1-O-Maloyl-(4,5-di-O-caffeoyl)quinic acid

| | | | |
|---|---|---|---|
| Maloyl | OH | Caffeoyl | Caffeoyl |

3-O-Maloyl-(4,5-di-O-caffeoyl)quinic acid

| | | | |
|---|---|---|---|
| OH | Maloyl | Caffeoyl | Caffeoyl |

the compounds of the family of CaffeoylMaloylTriCaffeoylQuinic acids (cm-TCQ), as described in table 5 below:

TABLE 5
CaffeoylMaloylTriCaffeoylQuinic Acid (cm-TCQ)

| | | | |
|---|---|---|---|
| Q1 | Q3 | Q4 | Q5 |

1-O-(2-Caffeoyl)maloyl-(3,4,5-tri-O-caffeoyl)quinic acid

| | | | |
|---|---|---|---|
| CaffeoylMaloyl | Caffeoyl | Caffeoyl | Caffeoyl |

3-O-(2-Caffeoyl)maloyl-(1,4,5-tri-O-caffeoyl)quinic acid

| | | | |
|---|---|---|---|
| Caffeoyl | CaffeoylMaloyl | Caffeoyl | Caffeoyl |

4-O-(2-Caffeoyl)maloyl-(1,3,5-tri-O-caffeoyl)quinic acid

| | | | |
|---|---|---|---|
| Caffeoyl | Caffeoyl | CaffeoylMaloyl | Caffeoyl |

5-O-(2-Caffeoyl)maloyl-(1,3,4-tri-O-caffeoyl)quinic acid

| | | | |
|---|---|---|---|
| Caffeoyl | Caffeoyl | Caffeoyl | CaffeoylMaloyl |

the compounds of the family of CaffeoylMaloylDiCaffeoylQuinic acids (cm-DCQ) as described in table 6 below:

TABLE 6
CaffeoylMaloylDiCaffeoylQuinic Acid (cm-DCQ)

| | | | |
|---|---|---|---|
| Q1 | Q3 | Q4 | Q5 |

4-O-(2-caffeoyl)maloyl-(1,3-di-O-caffeoyl)quinic acid

| | | | |
|---|---|---|---|
| Caffeoyl | Caffeoyl | CaffeoylMaloyl | OH |

5-O-(2-caffeoyl)maloyl-(1,3-di-O-caffeoyl)quinic acid

| | | | |
|---|---|---|---|
| Caffeoyl | Caffeoyl | OH | CaffeoylMaloyl |

3-O-(2-caffeoyl)maloyl-(1,4-di-O-caffeoyl)quinic acid

| | | | |
|---|---|---|---|
| Caffeoyl | CaffeoylMaloyl | Caffeoyl | OH |

5-O-(2-caffeoyl)maloyl-(1,4-di-O-caffeoyl)quinic acid

| | | | |
|---|---|---|---|
| Caffeoyl | OH | Caffeoyl | CaffeoylMaloyl |

3-O-(2-Caffeoyl)maloyl-(1,5-di-O-caffeoyl)quinic acid

| | | | |
|---|---|---|---|
| Caffeoyl | CaffeoylMaloyl | OH | Caffeoyl |

4-O-(2-caffeoyl)maloyl-(1,5-di-O-caffeoyl)quinic acid

| | | | |
|---|---|---|---|
| Caffeoyl | OH | CaffeoylMaloyl | Caffeoyl |

1-O-(2-caffeoyl)maloyl-(3,4-di-O-caffeoyl)quinic acid

| | | | |
|---|---|---|---|
| CaffeoylMaloyl | Caffeoyl | Caffeoyl | OH |

5-O-(2-caffeoyl)maloyl-(3,4-di-O-caffeoyl)quinic acid

| | | | |
|---|---|---|---|
| OH | Caffeoyl | Caffeoyl | CaffeoylMaloyl |

1-O-(2-caffeoyl)maloyl-(3,5-di-O-caffeoyl)quinic acid

| | | | |
|---|---|---|---|
| Maloyl | Caffeoyl | OH | Caffeoyl |

4-O-(2-caffeoyl)maloyl-(3,5-di-O-caffeoyl)quinic acid

| | | | |
|---|---|---|---|
| OH | Caffeoyl | CaffeoylMaloyl | Caffeoyl |

1-O-(2-caffeoyl)maloyl-(4,5-di-O-caffeoyl)quinic acid

| | | | |
|---|---|---|---|
| CaffeoylMaloyl | OH | Caffeoyl | Caffeoyl |

3-O-(2-caffeoyl)maloyl-(4,5-di-O-caffeoyl)quinic acid

| | | | |
|---|---|---|---|
| OH | CaffeoylMaloyl | Caffeoyl | Caffeoyl |

According to a more specific aspect of the present invention, in the composition (ES) as defined previously, the compound of formula (Ia) corresponding to formula (I) as defined previously and for which Q₁ represents the maloyl radical of formula (IIIb), Q₃ and Q₄, and Q₅, which are identical, represent the caffeoyl radical of formula (II).

According to a more specific aspect of the present invention, in the composition (ES) as defined previously, the compound of formula (Tb) corresponding to formula (I) as defined previously and for which Q₁ represents the caffeoylmaloyl radical of formula (IVa), Q₃ and Q₅, which are identical, represent the caffeoyl radical of formula (II) and Q₄ represents an —OH radical.

According to a more specific aspect of the present invention, in the composition (ES) as defined previously, the compound of formula (Ib) corresponding to formula (I) as defined previously and for which Q₁ represents the caffeoylmaloyl radical of formula (Vb), Q₃ and Q₅, which are identical, represent the caffeoyl radical of formula (II) and Q₄ represents an —OH radical.

According to a more specific aspect of the present invention, in the composition (ES) as defined previously, the compound of formula (Ic) is the compound of formula (IC₁) corresponding to formula (I) as defined previously and for which Q₁ and Q₅, which are identical, represent the caffeoyl radical of formula (II), Q₃ represents an —OH radical and Q₄ represents the caffeoylmaloyl radical of formula (Va).

According to a more specific aspect of the present invention, in the composition (ES) as defined previously, the compound of formula (Ic) is the compound of formula (Ic₁) corresponding to formula (I) as defined previously and for which Q₁ and Q₅, which are identical, represent the caffeoyl radical of formula (II), Q₃ represents an —OH radical and Q₄ represents the caffeoylmaloyl radical of formula (IVb).

According to a more specific aspect of the present invention, in the composition (ES) as defined previously, the compound of formula (Ic) is the compound of formula (Ic₂) corresponding to formula (I) as defined previously and for which Q₁ and Q₄, which are identical, represent the caffeoyl radical of formula (II), Q₃ represents an —OH radical and Q₅ represents the caffeoylmaloyl radical of formula (IVa).

According to a more specific aspect of the present invention, in the composition (ES) as defined previously, the compound of formula (Ic) is the compound of formula (Ic₂) corresponding to formula (I) as defined previously and for which Q₁ and Q₄, which are identical, represent the caffeoyl radical of formula (II), Q₃ represents an —OH radical and Q₅ represents the caffeoylmaloyl radical of formula (IVb).

According to a more specific aspect of the present invention, composition (C₁) as defined previously is characterized in that said composition (ES) comprises:
at least one compound of formula (Ia) corresponding to formula (I) for which Q₁ represents the maloyl radical of formula (IIIa) or of formula (IIIb) and Q₃ and Q₄ and Q₅, which are identical, each represent the caffeoyl radical of formula (II);
a compound of formula (Ib) corresponding to formula (I) for which Q₁ represents the caffeoylmaloyl radical of formula (IVa) or of formula (IVb), Q₃ and Q₅ each represent the caffeoyl radical of formula (II) and Q₄ represents a hydroxyl radical, and
at least one compound of formula (Ic) chosen from:
   the compound of formula (Ic₁) corresponding to formula (I) for which Q₁ and Q₅ each represent the caffeoyl radical of formula (II), Q₃ represents a hydroxyl radical and Q₄ represents the caffeoylmaloyl radical of formula (IVa) or of formula (IVb); and
   the compound of formula (Ic₂) corresponding to formula (I) for which Q₁ and Q₄ represent the caffeoyl radical of formula (II), Q₃ represents a hydroxyl radical and Q₅ represents the caffeoylmaloyl radical of formula (IVa) or of formula (IVb).

According to another particular aspect of the present invention, the organic solvent (OS₁) present in composition (C₁) as defined previously is chosen from 1,2-propanediol, 1,3-propanediol and 2-methyl-2,4-pentanediol; it is more particularly 1,2-propanediol.

Composition (C₁) that is the subject of the present invention may be prepared by simple mixing of its constituents, at a temperature of between 20° C. and 60° C., more particularly between 20° C. and 40° C. and even more particularly between 20° C. and 30° C., and with mechanical stirring of anchor type at a speed of between 50 revolutions/minute and 150 revolutions/minute.

According to a second aspect, a subject of the invention is a process for preparing composition (C₁) as defined in any one of claims 1 to 3, comprising the following successive steps:
a step a) of cultivation under soilless conditions of the plant Arctium lappa fed with a nutrient solution, so as to obtain a biomass (BM₁);
a step b) of immersion of the roots of said biomass (BM₁) obtained in the preceding step a) in a medium (S₁), such that the biomass (BM₁) mixture (S₁) ratio is between 0.5 kg/l and 1.5 kg/l, said medium (S₁) comprising, per 100% of its own mass, from 20% to 35% by mass of water, the pH of which has been adjusted to a value of between 1.5 and 3.5 by addition of a protic acid chosen from sulfuric acid, phosphoric acid and hydrochloric acid, and from 65% to 80% by mass of an organic solvent (OS₁) chosen from 1,2-propanediol, 1,3-propanediol, 1,4-butanediol, 1,3-butanediol, 1,2-butanediol, 2-methyl-2,4-pentanediol, 1,6-hexanediol, 1,8-octanediol or a mixture of these diols;
a step c) of separation of the roots of the biomass on conclusion of the treatment defined in step b), in order to isolate a liquid phase (L₁);
a step d) of immersion of the roots of said biomass (BM₂) resulting from step c) in said medium (S₁); in a biomass (BM₂)/mixture (S₁) ratio of between 0.1 kg/L and 1.5 kg/l;
a step e) of separation of said biomass (BM₂) on conclusion of the treatment defined in step d), in order to isolate a liquid phase (L₂);
a step f) of filtration of said liquid phase (L₃) obtained in step d), in order to isolate a liquid phase (L₃),
a step g) of mixing said liquid phases (L₁) and (L₃), then, if necessary, of addition of water and/or of said organic solvent (OS), so as to obtain the expected composition (C₁).

Step a) of cultivation under soilless (or aeroponic) conditions of the plant Arctium lappa is performed according to the standard conditions known to a person skilled in the art, and more particularly those concerning the influence of the content of nitrogen (2)(3) (4) (5) (6), and of the content of phosphorus and of potassium present in the culture medium. Step a) of cultivation under soilless conditions is thus performed by optimizing the nitrogen/phosphorus/potassium ratio present in the nutrient medium, and by optimizing the electrical conductivity parameter of such a nutrient medium.

Step a) is generally performed at a temperature of between 20° C. and 40° C., for a period of between 4 and 10 weeks, so as to obtain a biomass (BM₁), in a large amount, in particular at the roots; step a) is halted when growth of the biomass (BM₁) is no longer observed.

According to another particular aspect, during step b) of the process as defined previously, the mixture (S₁) comprising, per 100% of its own mass, from 23% by mass to 32% by mass and even more particularly from 26% by mass to 32% by mass of water, and from 68% by mass to 77% by mass and even more particularly from 68% by mass to 74% by mass of an organic solvent (OS₁) selected from the elements of the group consisting of 1,2-propanediol, 1,3-propanediol, 1-4-butanediol, 1,3-butanediol, 2-methyl-2,4-pentanediol, 1,6-hexanediol and 1,8-octanediol.

In step b) of the process for preparing composition (C₁) as defined previously, the pH of the water present in the mixture (S₁) is adjusted to a value of between 1.5 and 3.5 by adding a protic acid chosen from the elements of the group consisting of sulfuric acid, phosphoric acid and hydrochloric acid.

According to a more particular aspect, in step b) of the process as defined previously, the pH of the water present in the mixture (S₁) is brought to a value of between 1.5 and 3.0 and even more particularly between 1.5 and 2.5 by adding a protic acid chosen from the elements of the group consisting of sulfuric acid, phosphoric acid and hydrochloric acid.

According to an even more particular aspect, in step b) of the process as defined previously, the pH of the water present in the mixture (S₁) is brought to a value of between 1.5 and 2.5 by adding hydrochloric acid.

In step b) of the process for preparing composition (C₁) as defined previously, the roots of the biomass (BM₁) obtained on conclusion of step a) are placed in contact with the mixture (S₁) as described previously for a time of between 10 minutes and 60 minutes, at a temperature of between 20° C. and 30° C., with a biomass (BM₁)/mixture (S₁) ratio of between 0.5 kg/L and 1.5 kg/L.

According to a more particular aspect, in step b) of the process as defined previously, the roots of the biomass (BM₁) obtained on conclusion of step a) are placed in contact with the mixture (S₁) as described previously, more particularly by immersion, for a time of between 10 minutes and 45 minutes, more particularly between 10 minutes and 30 minutes, at a temperature of between 20° C. and 30° C., with a biomass (BM₁)/mixture (S1) ratio of between 0.5 kg/L and 1.5 kg/L, more particularly between 0.5 kg/L and 1.0 kg/L.

In step d) of the process for preparing composition (C₁) as defined previously, the biomass (BM₂) obtained on conclusion of step c) is placed in contact with the mixture (S₁) as described previously for a time of between 24 hours and 72 hours, at a temperature of between 20° C. and 30° C., with a biomass (BM₂)/mixture (S₁) ratio of between 0.1 kg/L and 1.5 kg/L.

According to a more particular aspect, in step d) of the process as defined previously, the roots of the biomass (BM₂) obtained on conclusion of step c) are placed in contact with the mixture (S₁) as described previously, more particularly by immersion, for a time of between 24 hours and 60 hours, more particularly between 24 hours and 48 hours, at a temperature of between 20° C. and 30° C., with a biomass (BM2)/mixture (S₁) ratio of between 0.1 kg/L and 1.0 kg/L, and more particularly between 0.3 kg/L and 1.0 kg/L.

In step f) of the process for preparing composition (C₁) as defined previously, the filtration of the liquid (L₂) obtained on conclusion of step d), performed to recover a liquid (L₃), is performed with the devices and equipment, known to a person skilled in the art, for efficiently separating solids and liquids.

In step g) of the process for preparing composition (C₁) as defined previously, the mixing of the liquid (L₁) obtained on conclusion of step c) and of the liquid (L₃) obtained on conclusion of step f) is performed at a temperature of between 20° C. and 30° C., with mechanical stirring of anchor type at a speed of between 50 revolutions/minute and 150 revolutions/minute. If necessary, an optional addition of water or of organic solvent (OS₁) is performed so as to obtain the expected composition (C₁).

According to a particular aspect, in step g) of the process as defined previously, the content of organic solvent (OS₁) is adjusted to between 65% and 75% by mass and the water content is adjusted to 25% to 35% by mass, more particularly by adding water and/or organic solvent (OS₁)

According to another particular aspect of the present invention, the organic solvent (OS₁) is selected from the elements of the group consisting of 1,2-propanediol, 1,3-propanediol and 2-methyl-2,4-pentanediol.

According to another more particular aspect of the present invention, the protic acid present in the water of the mixture (S₁) used in step b) and in step d) of the process for preparing said composition (C₁) is phosphoric acid.

According to another aspect, a subject of the invention is the use of composition (C₁) as defined previously, as a moisturizing cosmetic active agent.

A subject of the invention is also a cosmetic formulation for topical use comprising at least one cosmetically acceptable excipient and an effective amount of composition (C₁) as defined previously.

The expression “for topical use” used in the definition of the cosmetic formulation used in the cosmetic process which is a subject of the present invention means that said formulation is used by application to the skin, whether it is a direct application in the case of a cosmetic formulation, or an indirect application when the cosmetic formulation according to the invention is impregnated onto a support intended to be brought into contact with the skin (paper, wipe, textile, transdermal device, etc.).

Said composition (C₂) is generally spread over the surface of the skin to be treated and the skin is then massaged for a few moments.

The expression “cosmetically acceptable” used in the definition of the cosmetic formulation for topical use, used in the cosmetic process which is a subject of the present invention, means, according to the Council of the European Economic Community Directive No. 76/768/EEC of Jul. 27, 1976, amended by Directive No. 93/35/EEC of Jun. 14, 1993, that said formulation comprises any substance or preparation intended to be brought into contact with the various parts of the human body (epidermis, bodily hair and head hair system, nails, lips and genitals) or with the teeth and oral mucosae, for the purpose, exclusively and mainly, of cleansing them, fragrancing them, modifying the appearance thereof and/or correcting body odors thereof and/or protecting them or keeping them in good condition.

The term “effective amount of a composition (C₁) as defined previously and present in the cosmetic formulation for topical use used in the process that is the subject of the present invention” means, for 100% of the mass of said cosmetic formulation for topical use, a proportion of between 0.01% and 5% by mass, more particularly between 0.01% and 3% by mass, even more particularly between 0.1% and 3% by mass and even more particularly between 0.5% and 2% by mass of composition (C₁).

The cosmetic formulations for topical use used in the cosmetic process that is the subject of the present invention are generally in the form of aqueous or aqueous-alcoholic or water-glycol solutions, in the form of a suspension, an emulsion, a microemulsion or a nanoemulsion, whether they are of water-in-oil, oil-in-water, water-in-oil-in-water or oil-in-water-in-oil type, or in the form of a powder.

The cosmetic formulations for topical use used in the cosmetic process that is the subject of the present invention may be packaged in a bottle, in a device of pump-action bottle type, in pressurized form in an aerosol device, in a device equipped with a perforated wall such as a grate or in a device equipped with a ball applicator (“roll-on” device).

In general, composition (C₁) present in the cosmetic formulations for topical use used in the cosmetic process that is the subject of the present invention is combined with chemical additives normally used in the field of formulations for topical use, such as foaming and/or detergent surfactants, thickening and/or gelling surfactants, thickeners and/or gelling agents, stabilizers, film-forming compounds, solvents and cosolvents, hydrotropic agents, spring or mineral waters, plasticizers, emulsifiers and coemulsifiers, opacifiers, nacreous agents, superfatting agents, sequestrants, chelating agents, oils, waxes, antioxidants, fragrances, essential oils, preserving agents, conditioning agents, deodorants, active principles intended to provide a treating and/or protective action with respect to the skin or the hair, sunscreens, mineral fillers or pigments, particles which provide a visual effect or which are intended for encapsulating active agents, exfoliating particles, texturing agents, optical brighteners and insect repellents.

As examples of foaming and/or detergent surfactants that may be combined with said composition (C₁), mention may be made of anionic, cationic, amphoteric or nonionic foaming and/or detergent surfactants;
among the foaming and/or detergent anionic surfactants are, for example, salts of alkali metals, of alkaline-earth metals, of ammonium, of amines or of amino alcohols, of alkyl ether sulfates, of alkyl sulfates, of alkylamido ether sulfates, of alkylarylpolyether sulfates, of monoglyceride sulfates, of α-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkyl sulfoacetates, of alkyl sarcosinates, of acylisethionates, of N-acyl taurates, of acyl lactylates, of N-acyl derivatives of amino acids, of N-acyl derivatives of peptides, of N-acyl derivatives of proteins or of N-acyl derivatives of fatty acids.
among the foaming and/or detergent amphoteric surfactants are, for example, alkylbetaines, alkylamidobetaines, sultaines, alkylamidoalkylsulfobetaines, imidazoline derivatives, phosphobetaines, amphopolyacetates and amphopropionates.
among the foaming and/or detergent cationic surfactants are, for example, quaternary ammonium derivatives.
among the foaming and/or detergent nonionic surfactants are, for example, alkylpolyglycosides including a linear or branched, saturated or unsaturated aliphatic radical, and including from 8 to 16 carbon atoms, for instance octyl polyglucoside, decyl polyglucoside, undecylenyl polyglucoside, dodecyl glucoside, tetradecyl polyglucoside, hexadecyl polyglucoside, 1,12-dodecanediyl polyglucoside; ethoxylated hydrogenated castor oil derivatives, for instance the product sold under the INCI name PEG-40 hydrogenated castor oil; polysorbates, for instance Polysorbate 20, Polysorbate 40, Polysorbate 60, Polysorbate 70, Polysorbate 80 and Polysorbate 85; coconut kernel amides; N-alkylamines.

Examples of thickening and/or gelling surfactants that may be combined with said composition (C₁) include optionally alkoxylated alkylpolyglycoside fatty esters, for instance ethoxylated methylpolyglucoside esters, such as the PEG 120 methyl glucose trioleate and the PEG 120 methyl glucose dioleate sold, respectively, under the names Glutamate™ LT and Glutamate™ DOE120; alkoxylated fatty esters, such as the PEG 150 pentaerythrityl tetrastearate sold under the name Crothix™ DS53, the PEG 55 propylene glycol oleate sold under the name Antil™ 141; fatty-chain polyalkylene glycol carbamates, for instance the PPG-14 laureth isophoryl dicarbamate sold under the name Elfacos™ T211, the PPG-14 palmeth-60 hexyl dicarbamate sold under the name Elfacos™ GT2125.

Examples of thickeners and/or gelling agents that may be combined with said composition (C₁) include linear or branched or crosslinked polymers of polyelectrolyte type, such as the partially or totally salified acrylic acid homopolymer, the partially or totally salified methacrylic acid homopolymer, the partially or totally salified 2-methyl[(1-oxo-2-propenyl)amino]-1-propanesulfonic acid (AMPS) homopolymer, copolymers of acrylic acid and of AMPS, copolymers of acrylamide and of AMPS, copolymers of vinylpyrrolidone and of AMPS, copolymers of AMPS and of (2-hydroxyethyl) acrylate, copolymers of AMPS and of (2-hydroxyethyl) methacrylate, copolymers of AMPS and of hydroxyethylacrylamide, copolymers of AMPS and of N,N-dimethylacrylamide, copolymers of AMPS and of tris(hydroxymethyl)acrylamidomethane (THAM), copolymers of acrylic or methacrylic acid and of (2-hydroxyethyl) acrylate, copolymers of acrylic or methacrylic acid and of (2-hydroxyethyl) methacrylate, copolymers of acrylic or methacrylic acid and of hydroxyethylacrylamide, copolymers of acrylic or methacrylic acid and of THAM, copolymers of acrylic or methacrylic acid and of N,N-dimethylacrylamide, terpolymers of acrylic or methacrylic acid, of AMPS and of (2-hydroxyethyl) acrylate, terpolymers of acrylic or methacrylic acid, of AMPS and of (2-hydroxyethyl) methacrylate, terpolymers of acrylic or methacrylic acid, of AMPS and of THAM, terpolymers of acrylic or methacrylic acid, of AMPS and of N,N-dimethylacrylamide, terpolymers of acrylic or methacrylic acid, of AMPS and of acrylamide, copolymers of acrylic acid or methacrylic acid and of alkyl acrylates, the carbon chain of which comprises between 4 and 30 carbon atoms and more particularly between 10 and 30 carbon atoms, copolymers of AMPS and of alkyl acrylates, the carbon chain of which comprises between 4 and 30 carbon atoms and more particularly between 10 and 30 carbon atoms, linear, branched or crosslinked terpolymers of at least one monomer bearing a free, partially salified or totally salified strong acid function, with at least one neutral monomer, and at least one monomer of formula (VIII):

CH₂═C(R₃)—C(═O)—[CH₂—CH₂—O]ₙ—R₄ (VIII)

in which R₃ represents a hydrogen atom or a methyl radical, R4 represents a linear or branched alkyl radical including from 8 to 30 carbon atoms and n represents a number greater than or equal to 1 and less than or equal to 50.

Said polymers may be in the form of a solution, an aqueous suspension, a water-in-oil emulsion, an oil-in-water emulsion or a powder. Examples of commercial polymers include those sold under the names Simulgel™ EG, Simulgel™ EPG, Sepigel™ 305, Simulgel™ 600, Simulgel™ NS, Simulgel™ INS100, Simulgel™ FL, Simulgel™ A, Simulgel™ SMS 88, Sepinov™ EMT10, Sepiplus™ 400, Sepiplus™ 265, Sepiplus™ S, Sepimax™ ZEN, Aristoflex™ AVC, Aristoflex™ AVS, Novemer™ EC-1, Novemer™ EC2, Aristoflex™ HMB, Cosmedia™ SP, Flocare™ ET25, Flocare™ ET75, Flocare™ ET26, Flocare™ ET30, Flocare™ ET58, Flocare™ PSD30, Viscolam™ AT64 and Viscolam™ AT100.

Other examples of thickeners and/or gelling agents that may be combined with said composition (C₁) include:
polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=⅕), locust bean gum (DS=¼), tara gum (DS=⅓), guar gum (DS=½) or fenugreek gum (DS=1);
polysaccharides consisting of monosaccharide derivatives, such as sulfated galactans and more particularly carrageenans and agar, uronans and more particularly algins, alginates and pectins, heteropolymers of monosaccharides and of uronic acids, and more particularly xanthan gum, gellan gum, acacia gum exudates and karaya gum exudates, glucosaminoglycans;
cellulose and derivatives thereof, methylcellulose, ethylcellulose, hydroxypropylcellulose, silicates, starch, hydrophilic starch derivatives, and polyurethanes.

Examples of stabilizers that may be combined with said composition (C₁) include monocrystalline waxes, and more particularly ozokerite, mineral salts such as sodium chloride or magnesium chloride, and silicone polymers such as polysiloxane polyalkyl polyether copolymers.

Examples of spring or mineral waters that may be combined with said composition (C₁) include spring or mineral waters having a mineralization of at least 300 mg/I, in particular Avene water, Vittel water, Vichy basin water, Uriage water, La Roche Posay water, La Bourboule water, Enghien-les-bains water, Saint-Gervais-les-bains water, Neris-les-bains water, Allevard-les-bains water, Digne water, Maizieres water, Neyrac-les-bains water, Lons le Saunier water, Rochefort water, Saint Christau water, Fumades water and Tercis-les-bains water.

Examples of hydrotropic agents that may be combined with said composition (C₁) include xylene sulfonates, cumene sulfonates, hexyl polyglucoside, (2-ethylhexyl) polyglucoside and n-heptyl polyglucoside.

Examples of deodorants that may be combined with said composition (C₁) include alkali metal silicates, zinc salts, such as zinc sulfate, zinc gluconate, zinc chloride or zinc lactate; quaternary ammonium salts, such as cetyltrimethylammonium salts or cetylpyridinium salts; glycerol derivatives, such as glyceryl caprate, glyceryl caprylate or polyglyceryl caprate; 1,2-decanediol, 1,3-propanediol, salicylic acid, sodium bicarbonate, cyclodextrins, metal zeolites, Triclosan™, aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, or complexes of aluminum chlorohydrate and of glycol, such as the aluminum chlorohydrate and propylene glycol complex, the aluminum dichlorohydrate and propylene glycol complex, the aluminum sesquichlorohydrate and propylene glycol complex, the aluminum chlorohydrate and polyethylene glycol complex, the aluminum dichlorohydrate and polyethylene glycol complex or the aluminum sesquichlorohydrate and polyethylene glycol complex.

Examples of solvents and cosolvents that may be combined with said composition (C₁) include water, ethylene glycol, propylene glycol, butylene glycol, hexylene glycol, diethylene glycol, water-soluble alcohols such as ethanol, isopropanol or butanol, and mixtures of water and of said solvents.

Examples of oils that may be combined with said composition (C₁) include mineral oils such as liquid paraffin, liquid petroleum jelly, isoparaffins or white mineral oils; oils of animal origin such as squalene or squalane; plant oils, such as phytosqualane, sweet almond oil, coconut kernel oil, castor oil, jojoba oil, olive oil, rapeseed oil, groundnut oil, sunflower oil, wheat germ oil, corn germ oil, soybean oil, cotton oil, alfalfa oil, poppy oil, pumpkin oil, evening primrose oil, millet oil, barley oil, rye oil, safflower oil, candlenut oil, passionflower oil, hazelnut oil, palm oil, shea butter, apricot kernel oil, beauty-leaf oil, sisymbrium oil, avocado oil, calendula oil, oils derived from flowers or vegetables, ethoxylated plant oils; synthetic oils, for instance fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, fatty acid monoglycerides, diglycerides and triglycerides, for instance glyceryl triheptanoate, alkylbenzoates, hydrogenated oils, poly(α-olefins), polyolefins such as poly(isobutane), synthetic isoalkanes, for instance isohexadecane, isododecane, perfluorinated oils; silicone oils, for instance dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluoro groups, cyclic silicones and silicones modified with alkyl groups. In the present patent application, the term “oils” refers to compounds and/or mixtures of compounds which are water-insoluble, and which have a liquid appearance at a temperature of 25° C.

Examples of waxes that may be combined with said composition (C₁) include beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; plant waxes; fatty alcohols and fatty acids that are solid at room temperature; glycerides that are solid at room temperature. In the present patent application, the term “waxes” refers to compounds and/or mixtures of compounds which are water-insoluble, and which have a solid appearance at a temperature of greater than or equal to 45° C.

Examples of fatty substances that may be combined with said composition (C₁) include saturated or unsaturated, linear or branched fatty alcohols including from 8 to 36 carbon atoms, or saturated or unsaturated, linear or branched fatty acids including from 8 to 36 carbon atoms.

Examples of sunscreens that may be combined with said composition (C₁) include all those featured in the Cosmetic Directive 76/768/EEC, amended, Annex VII: notably the following compounds:
2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; the polysiloxane family, such as benzylidene siloxane malonate; or those of the following families of compounds:
the family of benzoic acid derivatives, such as para-aminobenzoic acids (PABAs), notably monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA, butyl esters of N,N-dimethyl PABA;
the family of anthranilic acid derivatives, such as homomenthyl-N-acetyl anthranilate;
the family of salicylic acid derivatives, such as amyl salicylate, homomenthyl salicylate, ethylhexyl salicylate, phenyl salicylate, benzyl salicylate, p-isopropylphenyl salicylate;
the family of cinnamic acid derivatives, such as ethylhexyl cinnamate, ethyl-4-isopropyl cinnamate, methyl-2,5-diisopropyl cinnamate, p-methoxypropyl cinnamate, p-methoxyisopropyl cinnamate, p-methoxyisoamyl cinnamate, p-methoxyoctyl cinnamate (p-methoxy 2-ethylhexyl cinnamate), p-methoxy 2-ethoxyethyl cinnamate, p-methoxycyclohexyl cinnamate, ethyl-α-cyano-β-phenyl cinnamate, 2-ethylhexyl-α-cyano-β-phenyl cinnamate, glyceryl di-para-methoxy mono-2-ethylhexanoyl cinnamate;
the family of benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone 5-sulfonate, 4-phenylbenzophenone, 2-ethylhexyl-4′-phenylbenzophenone-2-carboxylate, 2-hydroxy-4-(octyloxy)benzophenone, 4-hydroxy-3-carboxybenzophenone; 3-(4′-methylbenzylidene)-d,l-camphor, 3-(benzylidene)-d,l-camphor, benzalkonium methosulfate camphor;
the family of urocanic acid derivatives, such as this acid or ethyl urocanate;
the family of sulfonic acid derivatives, such as 2-phenylbenzimidazole-5-sulfonic acid and salts thereof,
the family of triazine derivatives, such as hydroxyphenyltriazine, (ethylhexyloxyxhydroxy)phenyl-4-methoxyphenyltriazine, 2,4,6-trianillino(p-carbo-2′-ethylhexyl-1′-oxy)-1,3,5-triazine, 4,4-((6-(((1,1-dimethylethyl)amino)carbonyl)phenyl)amino)-1,3,5-triazine-2,4-diyldiimino)bis-(2-ethylhexyl) ester of benzoic acid;
the family of diphenylacrylate derivatives, such as 2-ethylhexyl 2-cyano-3,3-diphenyl-2-propenoate or ethyl 2-cyano-3,3-diphenyl-2-propenoate.

Among the mineral sunscreens, also known as “mineral sunblocks”, that may be combined with said composition (C₁) are titanium oxides, zinc oxides, cerium oxide, zirconium oxide, yellow, red or black iron oxides, and chromium oxides. These mineral sunblocks may or may not be micronized, may or may not have undergone surface treatments and may optionally be in the form of aqueous or oily predispersions.

A subject of the invention is also a for improving the state of moisturization of the epidermis of human skin, characterized in that it comprises at least one tp a) of applying to the surface of the skin to be treated, an effective amount of the composition (C₂) for topical use as defined previously.

For the purposes of the present patent application, the term “improving the state of moisturization of the epidermis of human skin” means increasing the degree of moisturization of the stratum corneum of the epidermis of human skin, measured and observed within a period of between 7 days and 21 days after the application of said composition for topical use (C₂), as defined previously, onto the surface of said stratum corneum of the epidermis of human skin, of greater than or equal to 25% relative to the degree of moisturization measured and observed before application of said composition for topical use (C₂) to the surface of said stratum corneum of the epidermis of the human skin to be treated.

In the definition of the process as defined above, the term “effective amount” denotes an amount such that the state of moisturization of the stratum corneum of the epidermis of the human skin obtained after application to the epidermis of the skin to be treated shows an increase in the degree of moisturization of the stratum corneum of the epidermis of the treated human skin of greater than 25% relative to the degree of moisturization measured before application of the topical composition (C₂) as defined previously to the surface of the epidermis of the skin to be treated, after a period of between 7 days and 21 days after application of said composition (C₂) to said surface of the epidermis to be treated.

A subject of the invention is also a composition (C₁) as defined previously, for its use in a therapeutic treatment for reducing and/or eliminating and/or preventing chapping and/or dry patches and/or cracks and/or atopic dermatitis and/or ichthyosis and/or dryness of the skin or the mucosae accompanying cutaneous and/or mucosal pathologies such as eczema.

Composition (C₁), for its use in a therapeutic treatment as defined previously, may be combined with pharmaceutical, in particular dermatological, active ingredients.

### BIBLIOGRAPHY

(1): Chan et al., “A review of the pharmacological effects of Artium lappa”, Inflammopharmacol., 2011, 19, 245-254).
(2). Rasmussen, S., Parsons, A. J., Fraser, K., Xue, H., and Newman, J. A. (2008): “Metabolic profile of lolium flowers and burdock roots using a standardized LC-DAD-ESI/MS profiling method”, Journal of Agricultural and Food Chemistry 56, 10105-10114.
(3): Dornenburg, and Knorr (1995), “Strategies secondary for improvement of metabolite production in plant cell cultures”, Enzyme and Microbial Technology, 17, 674-684.
(4): Dinh, P. T. Y., Roldan, M., Leung, S., and McManus, M. T (2012), “Regulation of root growth by auxin and ethylene is influenced by phosphate supply in white clover (Trifollium repens L.)”, Plant Growth Regulation, 66, 179-180.
(5): Badry, D. V., and Vivanco, J. M. (2009), “Regulation and function of root exudates”, Plant, Cell & Environment 32, 666-681
(6): Baenas N., Garcia-Viguera G., and Moreno D. A. (2014), “Elicitation: a tool for enriching the bioactive composition of foods”, Molecules 19, 13541-13563.

### DESCRIPTION OF THE PREFERRED EMBODIMENTS

The examples that follow illustrate the invention without, however, limiting it.

A₁) -Preparation of a Composition (C_{1A}) According to the Invention

Burdock or Arctium lappa plants were obtained beforehand by germination of seeds over a period of 60 days under standard conditions, so as to reach a size of about 10 to 15 cm, then they are removed from their pots and placed under aeroponic culture conditions, consisting in leaving the burdock plants to soak in a nutrient solution characterized by an electrical conductance of between 1.0 mS (millisiemens) and 1.2 mS and by an N/P/K (nitrogen/phosphorus/potassium) mass ratio provided by the fertilizer of about 15/10/30; for 6 weeks at a temperature regulated at 20° C. A root yield of 754 g per square meter is thus obtained.

The fresh roots of the biomass thus obtained are collected and immersed for 15 minutes at 25° C., at a rate of 1.0 kg of biomass per liter, in a bath comprising, per 100% of its mass, 70% by mass of 1,2-propanediol and 30% by mass of distilled water, the pH of which was adjusted beforehand to 2.0±0.2, by adding a solution containing 75% by mass of phosphoric acid.

The plants are then removed from their exudation bath (L₁), the roots are drained and then chopped up, and the remaining biomass is then macerated again for 48 hours at 25° C., at a rate of 0.5 kg of biomass per liter, in a new bath comprising a mixture comprising, per 100% of its mass, 70% by mass of 1,2-propanediol and 30% by mass of distilled water, the pH of which was adjusted beforehand to 2.0±0.2, by adding a solution containing 75% by mass of phosphoric acid.

On conclusion of this maceration phase, the biomass is separated from the maceration liquid (L₂) and said liquid (L₂) is filtered with a 50 micrometer filter bag.

The liquids (L₁) and (L₂) are pooled and the mixture obtained is then supplemented with 1,2-propanediol to adjust its mass content to 70%. The liquid (La) thus obtained is filtered through a 1 micrometer membrane so as to clarify it, and then through a 0.2 micrometer membrane to obtain the expected composition (C_{1A}).

A₂) -Example of Preparation of a Comparative Composition (C_{comp})

The plantlets resulting from the same batch of seeds as that used to obtain the plantlets subsequently cultivated aeroponically (example A₁) are used for cultivation of said plantlets in soil for a period of time of six weeks.

At the end of this period, the plants are removed from the pots, the fresh roots are cleaned, cut up and ground, and said ground material obtained is then extracted according to a conventional liquid/solid extraction process (maceration, agitation, filtration) using a 70/30 1,2-propanediol/distilled water solvent mixture, at a temperature of 25° C., with a fresh roots/solvent medium mass ratio of 0.5 kg of biomass per 1 liter of 1,2-propanediol and water mixture; the value of the pH of the distilled water having been adjusted beforehand to 2.0±0.2 by addition of a solution containing 75% by mass of phosphoric acid.

At the end of this extraction phase, the biomass is separated from the liquid, which is subsequently filtered with a 50 micrometer filter bag, then with a 1 micrometer membrane, so as to clarify it, and finally under sterilizing filtration with a 0.2 micrometer membrane, so as to achieve the composition (C_{comp}).

A₃) Analytical Characterization of the Composition (C_{1A}) According to the Invention and of the Comparative Composition (C_{comp})

The characteristics of compositions (C₁) (C_{comp}) are collated in table 11 below:

TABLE 11

| | | Composition | |
|---|---|---|---|
| Characteristics | Analytical method | (C_{1A}) | (C_{comp}) |
| Appearance | Visual | Yellow | Yellow |
| | | Liquid | Liquid |
| 1,2-Propanediol content | GC (headspace) | 71.4% | 70.0% |
| pH | According to the | 3.3 | 3.2 |
| | standard NFT | | |
| | 73-206 | | |
| Solids content as % | Oven at 105° C., | 0.21% | 1.12% |
| by mass | 12 hours | | |
| Water % by mass | According to the | 28.39% | 28.88% |
| | standard NFT 73201 | | |
| Total content of compounds | UPLC-MS* | 613.3 | 169.5 |
| of formulae (Ia), (Ib), (Ic₁) | | | |
| and (Ic₂) (mg/gram | | | |
| of solids content) | | | |

*UPLC-MS: Machine: Shimadzu Nexera X2; Column: Waters Xterra RP C18; 250 × 4.6 mm; mobile phase (with gradient): water + formic acid/acetonitrile; UV detector (330 nm)

B) Demonstration of the Moisturizing Properties of Composition (C_{1A}) According to the Invention and of the Comparative Composition (C_{comp}).

### B₁) Demonstration of the Protection Against Impairment of the Skin's Barrier Function, on Samples of Reconstructed Human Epidermis.

### B1.1. Principle of the Method

The strains Staphylococcus epidermidis and Staphylococcus aureus were cultured in BHI(Brain Heart Infusion) and NB(Nutrient Broth) medium, respectively, at 37° C. for 24 hours. Samples of reconstructed human epidermis with a surface area of 0.5 cm², cultivated at 37° C. and under 5% CO₂, were colonized first with the strain Staphylococcus epidermidis for a period of 6 hours, and were then colonized with the strain Staphylococcus aureus for 24 hours.
Composition (C_{1A}) according to the invention (1% v/v) was added to the samples of reconstructed human epidermis at the same time as the strain Staphylococcus epidermidis and then again with the strain Staphylococcus aureus.
The barrier function of the samples of reconstructed human epidermis thus treated was evaluated:

by measurement of the transepithelial electrical resistance (TEER) of the samples of reconstructed human epidermis and

by histological evaluation of said samples of reconstructed human epidermis, and more particularly by staining with hematoxylin and eosin, and by a “score” of said staining.

In the context of the histological evaluation, the effects on the barrier function of the samples of reconstructed human epidermis were evaluated by a histological score on staining with hematoxylin and eosin, which was attributed as follows:

0=standard: no significant modification of the reference morphology

1=mild: significant modification of the stratum corneum

2=moderate: significant modifications of the stratum corneum and of the granular layer, decrease in keratohyalin and a few necrotic cells

3=strong: significant modifications in the basal layer with necrotic cells and intercellular holes and edemas

4=severe: loss of intercellular connection, detachment of the tissue from the polycarbonate filter, necrotic cells, absence of specific labeling.

A product is judged to be protective on the barrier function of human epidermis if the histological score is graded “standard” (score 0) or “mild” (score 1).

The equilibrium of the microbiota of the reconstructed human epidermis, with or without application of composition (C_{1A}), was evaluated by studying the formation of ultrastructures such as colonies and biofilms by SEM.

### B.1.2. Results

B.1.2.1 Results obtained on the protection of the barrier function of the samples of reconstructed human epidermis by measuring the transepithelial electrical resistance (TEER)

The TEER measurements performed on the samples of reconstructed human epidermis, as a function of the associated treatments, are collated in Table 7. A decrease in the transepithelial electrical resistance (TEER) is evidence of degradation of the barrier function of the epidermis and consequently constitutes one of the factors for skin dehydration and of the unesthetic effects that this dehydration may cause. The difference A between the transepithelial electrical resistance of the surface of the reconstructed human epidermis after colonization and before colonization is also calculated. The percentage of protection is also calculated according to the formula: % Protection=R_{[reconstructed human epidermis colonized with Staphylococcus epidermidis+Staphylococcus aureus and treated with (C1A) 1% (v/v)]}- R_{[reconstructed human epidermis colonized with Staphylococcus epidermidis+Staphylococcus aureus without addition of (C1A)]}/R_{[untreated reconstructed human epidermis (control)]}−R_{[reconstructed human epidermis colonized with Staphylococcus epidermidis+Staphylococcus aureus without addition of (C1A)]}

TABLE 7
Untreated reconstructed human epidermis (control)

| | At t = 0 |
|---|---|
| Electrical resistance (in Ohm · cm²) | R₀ = 8073.33 ± 1913.60 |
| | At t = 30 hours |
| Electrical resistance (in Ohm · cm²) | R′₀ = 6900.00 ± 1489.80 |
| Δ₀ = R′₀ − R₀ | −1173.33 |

Reconstructed human epidermis colonized with Staphylococcus
epidermidis Staphylococcus aureus without addition of
Composition (C_{1A})

| | |
|---|---|
| | Before colonization (t = 0) |
| Electrical resistance (in Ohm · cm²) | R₁ = 8094.44 ± 1486.73 |
| | After colonization (t = 30 hours) |
| Electrical resistance (in Ohm · cm²) | R′₁ = 4726.11 ± 2067.16 |
| Δ₁ = R′₁ − R₁ | −3368.33 |
| (100/Δ₀) × (Δ₁ − Δ₀) | 187% |

Reconstructed human epidermis colonized with Staphylococcus
epidermidis + Staphylococcus aureus and treated with Composition
(C_{1A}) 1% (v/v) (0.0136% of dry extract of composition (C_{1A}))

| | |
|---|---|
| | Before colonization |
| Electrical resistance (in Ohm · cm²) | R₂ = 7647.22 ± 1566.61 |
| | After colonization |
| Electrical resistance (in Ohm · cm²) | R′₂ = 5645.56 ± 911.52 |
| Δ₂ = R′₂ − R₂ | −2001.66 |
| (100/Δ₀) × (Δ₂ − Δ₀) | 71% |
| % of protection [(R′₂ − R′₁)/(R′₀ − R′₁)] | 42% |

When the samples of reconstructed human epidermis are colonized with Staphylococcus epidermidis and Staphylococcus aureus, the difference in TEER measured before and after the start of said colonization is −3368.33 Ohm·cm², and shows an increase of 187% relative to the samples of non-colonized and untreated reconstructed human epidermis (−1173.33 Ohm·cm²).

When the samples of reconstructed human epidermis are placed in contact with composition (C_{1A}) at the same time as their colonization with Staphylococcus epidermidis and Staphylococcus aureus, the difference between the TEER values measured before and after said colonization is −2001.66 Ohm·cm², which represents an increase of 71% relative to the samples of non-colonized and untreated reconstructed human epidermis (−1173.33 Ohm·cm²) and a protection of 42% relative to the samples of reconstructed human epidermis colonized with Staphylococcus epidermidis and Staphylococcus aureus.

As a result, application to the skin of a composition comprising composition (C_{1A}) makes it possible to prevent the degradation of the barrier function of the epidermis of the skin, before the skin is subjected to the action of bacteria known for their degrading effects on the barrier function of said epidermis of the skin.

B.1.2.2 Results obtained on the protection of the barrier function of samples of reconstructed human epidermis by histological evaluation of said samples of reconstructed human epidermis, by staining with hematoxylin and eosin.

The staining with hematoxylin and eosin of the epidermal samples was evaluated by attributing a “score” as described previously, and the results are collated in table 8 below:

TABLE 8
Histological score

Untreated reconstructed human epidermis (control)
0: no significant modification vs the reference morphology
Human epidermis colonized with Staphylococcus epidermidis +
Staphylococcus aureus without addition of Composition (C_{1A})
2: modification of the structure of the viable epidermis with more
intercellular spaces and regrouping of cells.
Reconstructed human epidermis colonized with Staphylococcus
epidermidis + Staphylococcus aureus and treated
with Composition (C_{1A}) 1% (v/v) (0.0136% of dry extract due
to the plant of composition (C_{1A}))
1: reduction of the damage, in particular at the basal level and in the
stratum corneum where a more compact lamellar structure is observed

When the samples of reconstructed human epidermis were placed in contact with Composition (C_{1A}) at the same time as their colonization with Staphylococcus epidermidis and Staphylococcus aureus, the histological score was evaluated at a level of 1, which is evidence of a reduction of the damage, in particular at the basal level and in the stratum corneum where a more compact lamellar structure is observed.

As a result, the application to the skin of a composition comprising Composition (C_{1A}) makes it possible to prevent degradation of the tissue cohesion and consequently of the barrier function of the epidermis in the face of an invasion of the transient flora. Furthermore, the profile of colonization with the two bacteria (Staphylococcus epidermidis and Staphylococcus aureus) was evaluated by scanning electron microscopy (SEM, Zeiss Sigma Electron Microscope).

When the samples of reconstructed human epidermis were colonized only with Staphylococcus epidermidis, the bacterium is present homogeneously on the surface of the reconstructed human epidermis, forming large aggregates and developing a biofilm characterized by filamentous polysaccharide structures observed at a magnification of ×10 000 of the electron microscope.

When the samples of reconstructed human epidermis were colonized with Staphylococcus epidermidis and Staphylococcus aureus, the appearance of several spherical aggregates of Staphylococcus aureus on the surface of the reconstructed human epidermis is observed, whereas a film of Staphylococcus epidermidis remains visible on the surface of said epidermis.

The presence of large aggregates of Staphylococcus aureus is also observed, with a magnification of ×10 000 of the electron microscope, forming a three-dimensional structure, thus indicating an early stage of development of a biofilm at the surface of the epidermis.

After application to the skin of a composition comprising Composition (C_{1A}), the spherical aggregates of Staphylococcus aureus are no longer present, thus meaning that the addition of Composition (C_{1A}) makes it possible to prevent the adhesion of the bacteria to the surface of the epidermis and the formation of the Staphylococcus aureus biofilm. The Staphylococcus epidermidis biofilm still remains visible on the surface of the epidermis.

These observations show that Composition (C_{1A}) makes it possible to reduce the adhesion and thus the formation of biofilms of pathogenic opportunistic bacteria, for instance Staphylococcus aureus, without impairing the presence of commensal bacteria such as Staphylococcus epidermidis.

### B.1.3. Conclusions

The combination of measurement of the transepithelial electrical resistance and of the histological evaluation of samples of reconstructed human epidermis, before colonization with a commensal bacterium of the cutaneous flora, and then with a pathogenic bacterium, constitutes a model for studying the impairment of the barrier function and the equilibrium of the microbiota of said epidermis, and the incidence of prior treatments with compositions or extracts or complex formulations.

The set of results and observations collected in sections B.1.2.1 and B.1.2.2 shows that composition (C_{1A}) makes it possible to prevent degradation of the barrier function of the epidermis of human skin and consequently to prevent dehydration of the epidermis of the human skin in a model in which it is colonized with a commensal bacterium of the cutaneous flora, and then with a pathogenic bacterium, and the unesthetic effects associated therewith, for example dry, rough skin, which may be accompanied by inflammation and itching when it is associated with the presence of pathogenic bacteria.

### B₂) Demonstration of the Improvement in the Barrier Function of the Skin by Increasing the Keratinocyte Differentiation

### B.2.1 Principle of the Method

The evolution of differentiation of the epidermal keratinocytes constitutes a means for showing the effect of a treatment of said epidermis on the evolution of the barrier function of the epidermis of human skin, and consequently on the evolution of the state of moisturization of the epidermis of human skin.

To do this, normal human keratinocytes were cultured at 37° C. and 5% CO₂. After a period of 4 days, they were treated with the products whose action it is desired to evaluate.

After culturing for a further 72 hours at 37° C. and 5% CO₂ with the test products, the cell lawns were rinsed, dried and then fixed with glacial methanol. Filaggrin, a keratinocyte differentiation marker, was detected and quantified by fluorescent immunolabeling with an anti-filaggrin primary antibody and a fluorescent secondary antibody to reveal the former. The nuclei were detected and quantified by marking with Hoechst, a fluorescent stain which binds to DNA and thus labels the nuclei. This last labeling was performed to normalize the results obtained previously on filaggrin.

### B.2.2 Results

The filaggrin measurements taken on keratinocytes cultured under different conditions, and notably in the presence of Composition (C_{1A}) according to the invention and in the presence of the comparative composition (C_{1Com}), are collated in Table 9.

An increase in the filaggrin (area of labeling)/nuclei (area of labeling) ratio is evidence of an increase in the differentiation of the epidermal keratinocytes, and consequently of reinforcement of the barrier effect of the epidermis, which constitutes one of the factors of the reinforcement of the state of moisturization of the skin. An increase in keratinocyte differentiation makes it possible to reinforce the barrier effect of the human epidermis, and consequently to decrease the unesthetic effects that may be caused by the dehydration of human skin.

TABLE 9

| | |
|---|---|
| Filaggrin (area of labeling)/ | |
| nuclei (area of labeling) | % stimulation vs control |

Control medium (culture medium in the absence oftreatment composition)

| | |
|---|---|
| 0.0034 ± 0.0003 | — |

Medium treated with positive reference CaCl₂ 1 mMol/L

| | |
|---|---|
| 0.0245 ± 0.0045 | 621* |

Medium treat with Composition (C_{1A}) 0.08% by mass

| | |
|---|---|
| 0.0254 ± 0.0030 | 648 |

Medium treated with Composition (C_{1Comp}) 0.08% by mass

| Filaggrin (area of labeling)/ | % stimulation vs Composition |
|---|---|
| nuclei (area of labeling) | (C_{1A}) |
| 0.0144 ± 0.0059 | 323p = 0.06 |

### B.2.3. Conclusions

The application of Composition (C_{1A}) according to the invention makes it possible to achieve a filaggrin/nucleus ratio value of 0.0254, as opposed to 0.0144 when Composition (C_{1A}) is replaced with Composition (C_{1comp}). Thus, the application of Composition (C_{1A}) according to the invention makes it possible to increase the filaggrin production by 648% relative to the control (untreated keratinocytes) whereas comparative composition (C_{1Comp}) increases the filaggrin production by only 323%.

Composition (C_{1A}) according to the invention makes it possible to increase the keratinocyte differentiation, and consequently to reinforce the barrier effect of the epidermis, and consequently to promote the state of moisturization of said human epidermis.

B₃) Effect of Composition (C_{1A}) According to the Invention on the Degree of Moisturization of the Skin.

### B.3.1. Principle of the Method

The degree of skin moisturization is determined by evaluating the electrical properties of the skin, for instance the impedance, the resistance and the capacitance, since these dielectric parameters measured at the surface of the skin vary with the amount of water contained in the stratum corneum.

The principle adopted and used in the context of the present patent application is based on measurement of the variation in the dielectric capacitance of the surface of the skin. Specifically, just like any material or any biological matrix, the stratum corneum can be characterized by its mean capacitance value; this dielectric property varies with the amount of water it contains.

### B.3.2 Equipment

The degree of skin moisturization is measured using a CM825™ model corneometer, sold by the company Courage & Khasaka, equipped with a sensor composed of two metal electrodes. When the corneometer is supplied with electricity, it enables an electric field to be applied across the stratum corneum and measurement of the capacitance corresponding to the state of the skin onto which the electric field has been applied.

### B.3.3 Experimental Protocol

Study of the moisturizing effect of the test compositions was performed on a group of 21 volunteers, and consisted in applying twice a day the compositions to be evaluated, either the placebo (referred to herein as PLAC) or the test composition (referred to herein as COMP), or by leaving an untreated zone (referred to herein as NT), for 21 days. The measurements of the degree of skin moisturization measured using the corneometer were taken before applying the compositions (D0), after a period of seven days following the application of the test compositions to the skin (D7) and after a period of 21 days following the application of the test compositions to the skin (D21).

The characteristics of the group of volunteers were as follows:
women from 18 to 48 years old,
with an average age of 35,
and of phototype II to IV
and having very dry skin on the legs (≤30 (au) by corneometry on inclusion into the study).

### B.3.4 Expressing the Results

The measurements of the degree of moisturization are expressed in arbitrary units (au).

An increase in the values (expressed in au) indicates an increase in the water content of the stratum corneum, thus characterizing a moisturizing effect.

The values measured with the corneometer at each of the measuring times are recorded, and the variations between DO and D7 and then between DO and D21 are expressed as a percentage relative to the value measured at DO, for each composition to be evaluated.

The following are thus defined:
T_{(D0)}: The mean value of the degree of moisturization, expressed in arbitrary units (au), measured before applying the test compositions, on the area to be treated, and on the untreated area;
T_{(D7)}: The mean value of the degree of moisturization, expressed in arbitrary units (au), measured seven days after applying the test compositions, on the treated area, and on the untreated area;
T_{(D21)}: The mean value of the degree of moisturization, expressed in arbitrary units (au), measured 21 days after applying the test compositions, on the treated area, and on the untreated area;

Δ₇, the percentage increase in the degree of moisturization after seven days of treatment, is thus calculated:

For the placebo:

Δ₇=100×[(T_{(D7)}−T_{(D0)})_{PLAC}−(T_{(D7)}−T_{(D0)})_{NT}]/[T_{(D0)})_{PLAC}−(T_{(D7)}−T_{(D0)})_{NT}]

For the test composition:

Δ₇=100×[(T_{(D7)}−T_{(D0)})_{COMP}−(T_{(D7)}−T_{(D0)})_{NT}]/[T_{(D0)})_{COMP}−(T_{(D7)}−T_{(D0)})_{NT}]

Δ₂₁, the percentage increase in the degree of moisturization after 21 days of treatment, is also calculated:
For the placebo:

Δ₂₁=100×[(T_{(D21)}−T_{(D0)})_{PLAC}−(T_{(D21)}−T_{(D0)})_{NT}]/[T_{(D0)})_{PLAC}−(T_{(D21)}−T_{(D0)})_{NT}]

For the test composition:

Δ₂₁=100×[(T_{(D21)}−T_{(D0)})_{COMP}−(T_{(D21)}−T_{(D0)})_{NT}]/[T_{(D0)})_{COMP}−(T_{(D21)}−T_{(D0)})_{NT}]

The effect between the products is also calculated:
% vs placebo at D₇=

100×(T_{(D7)}−T_{(D0)})_{COMP}−(T_{(D7)}−T_{(D0)})_{PLAC}]/[T_{(D7)}−T_{(D0)})_{PLAC}−T_{(D7)}−T_{(D0)})_{NT}]

% vs placebo at D₂₁=

100×[(T_{(D21)}−T_{(D0)})_{COMP}−(T_{(D21)}−T_{(D0)})_{PLAC}]/[T_{(D21)}−T_{(D0)})_{PLAC}−T_{(D21)}−T_{(D0)})_{NT}]

### B.3.5 Results Obtained

The mean measurements of the degree of moisturization that were obtained for the application of the test compositions are indicated in table 10 below:

TABLE 10

| Product | Δ₇ | Δ₂₁ |
|---|---|---|
| Placebo | +18.3% | +22.2% |
| Formula containing Composition (C_{1A}) at 1% by mass | +28.8% | +37.9% |

The comparisons of the degrees of moisturization obtained between the placebo and the test composition at D7 and D21 are indicated in table 11 below:

TABLE 11

| | % vs placebo | |
|---|---|---|
| Product | at D 7 | at D 21 |
| Formula containing Composition (C_{1A}) at 1% by mass | +58% | +56% |

### B.3.6 Analysis of the Results

After 7 days following the application of the test compositions, the evolution of the mean of the measured degrees of moisturization Δ7, shows that the increase in the degree of moisturization is 28.8% for composition (C_{1A}) according to the invention as opposed to 18.8% for the placebo composition. The increase in the degree of moisturization for composition (C_{1A}) according to the invention is 58% versus placebo.

After 21 days following the application of the test compositions, the evolution of the mean of the measured degrees of moisturization Δ₂₁, shows that the increase in the degree of moisturization is 37.9% for composition (C_{1A}) according to the invention as opposed to 22.2% for the placebo composition. The increase in the degree of moisturization for composition (C_{1A}) according to the invention is 56% versus placebo.

As a result, composition (C_{1A}) according to the invention makes it possible to improve the degree of moisturization of human epidermis.

### B.4 Analysis

The set of results obtained in section B.3 of the present patent application demonstrates that Composition (C_{1A}) according to the invention affords a moisturizing effect on the epidermis of human skin, both via its role in reinforcing the barrier effect of the epidermis (protection against degradation, and increase) and via its action on increasing the degree of moisturization of the epidermis of human skin.

### C) Formulations

In the following formulas, the percentages are expressed by weight of the formulation.

### C.1) -Makeup-Removing Fluid for the Face

### Formula

| | |
|---|---|
| Composition (C_{1A}) | 10.00% |
| Methyl paraben | 0.15% |
| Phenoxyethanol | 0.80% |
| Sepicalm ™ S | 1.00% |
| Perfume/Fragrance | 0.10% |
| Water | qs 100.00% |

Procedure: Mix the various ingredients in the water with magnetic stirring, in the order indicated, and adjust the pH to about 7.

### C.2) -Infant Hair and Body Shampoo

### Formula

| A | Composition (C_{1A}) | 15.00% |
|---|---|---|
| Proteol ™ APL | | 5.00% |
| Sepicide ™ HB | | 0.50% |
| Perfume/Fragrance | | 0.10% |
| B | Water | 20.00% |
| Capigel ™ 98 | | 3.50% |
| C | Water | qs 100.00% |

| | |
|---|---|
| Sepicide ™ CI | 0.30% |
| Colorant | qs |
| Sodium hydroxide | qs pH = 7.2 |

Procedure: Mix composition (E₄) with the Proteol™ APL and the Sepicide™ HB (Phase A). Dilute the Capigel™ 98 in a portion of the water and add it to phase A obtained previously (Phase B). Add the rest of the water to phase B, followed by the Sepicide™ CI and the colorant. Adjust the pH of the mixture to about 7.2 with sodium hydroxide.

### C.3) -Makeup-Removing Wipes for the Eyes

### Formula

| A | Composition (C_{1A}) | 3.00% |
|---|---|---|
| B | Sepicide ™ HB2 | 0.50% |
| C | Sepicalm ™ VG | 0.50% |
| Perfume/Fragrance | | 0.05% |
| D | Water | qs 100.00% |

Procedure: Mix the ingredients of phase B and those of phase C in phase A until the solution is clear. Add phase D.

### C.4) -Mild Foaming Gel

### Formula

| A | Composition (C_{1A}) | 8.50% |
|---|---|---|
| Proteol ™ APL | | 3.00% |
| Euxyl ™ PE9010 | | 1.00% |
| Perfume/Fragrance | | 0.10% |
| B | Water | qs 100.00% |

| | |
|---|---|
| Lactic acid | qs pH = 6.0 |

Procedure: Dissolve the perfume and the preserving agent Euxyl™ PE9010 in the mixture composed of composition E₄ and the Proteol™ APL (phase A). Add the water and adjust the pH to about 6.0 with lactic acid.

### C.5) -Regular-Use Shampoo

### Formula

| A | Composition (C_{1A}) | 12.80% |
|---|---|---|
| Proteol ™ OAT | | 5.00% |
| Euxyl ™ PE9010 | | 1.00% |
| Perfume/Fragrance | | 0.30% |
| Water | | qs 100.00% |
| B | Montaline ™ C40 | 8.50% |

| | |
|---|---|
| Lactic acid | qs pH = 6.0 |

Procedure: Mix all the ingredients of phase A and, after homogenization, add the Montaline™ C40 and adjust the pH to about 6.0 with lactic acid.

### C.6 Ultra-Mild Baby Shampoo

### Formula

| | | |
|---|---|---|
| A | Composition (C_{1A}) | 10.00% |
| | Amisoft ™ CS-11 | 4.00% |
| | Perfume/Fragrance | 0.10% |
| | Sepicide ™ HB | 0.30% |
| | Sepicide ™ CI | 0.20% |
| | Water | qs 100.00% |
| B | Water | 20.00% |
| | Capigel ™ 98 | 3.50% |
| | Tromethamine | qs pH = 7.2 |

Procedure: Mix all the ingredients of phase A, in the order indicated, until a clear phase A is obtained. Separately, add the Capigel™ 98 to the water and then add this phase B thus prepared to phase A and adjust the pH to 7.2 using tromethamine.

### C.7 Baby Cleansing Milk

### Formula

| | | |
|---|---|---|
| A | Simulsol ™ 165 | 2.00% |
| | Montanov ™ 202 | 1.00% |
| | Lanol ™ 99 | 3.00% |
| | Dimethicone | 1.00% |
| | Isohexadecane | 3.00% |
| B | Water | qs 100.00% |
| C | Sepiplus ™ 400 | 0.30% |
| D | Composition (C_{1A}) | 6.35% |
| E | Sepicide ™ HB | 0.30% |
| | DMDM Hydantoin | 0.20% |
| | Perfume/Fragrance | 0.10% |

Procedure: Heat, separately, phases A and B constituted by mixing the various constituents. Add phase C to the hot fatty phase and make the emulsion by pouring in the aqueous phase; homogenize for a few minutes with vigorous stirring (by means of a rotor/stator turbomixer). Next, add phase D to the hot emulsion and cool the emulsion with moderate stirring down to room temperature. Add phase E at 40° C.

### C.8 Cleansing Powder Lotion for Sensitive Skin

### Formula

| | | |
|---|---|---|
| A | Lipacide ™ C8G | 0.95% |
| | Methyl paraben | 0.10% |
| | Ethyl paraben | 0.024% |
| | Propyl paraben | 0.0119% |
| | Butyl paraben | 0.024% |
| | Isobutyl paraben | 0.0119% |
| | Water | 20.00% |
| | Disodium EDTA | 0.10% |
| | Triethanolamine | 1.38% |
| B | Composition (C_{1A}) | 1.80% |
| | Perfume/Fragrance | 0.10% |
| C | Sepicalm ™ S | 0.28% |
| | Water | qs 100.00% |
| | Lactic acid | qs pH = 5.2 |
| D | Micropearl ™ M310 | 5.00% |

Procedure: Dissolve the ingredients of phase A in the water at 80° C. Separately, dissolve the fragrance in composition (E₄) to prepare phase B. Add the cooled phase A to phase B, then introduce the Sepicalm™ S and the remaining water. Check the final pH and adjust to about 5.2 if necessary. Next, add the Micropearl™ M310.

### C.9 Infant Shower Gel

### Formula

| | | |
|---|---|---|
| A | Water | 56.06% |
| | Sepimax ™ Zen | 3.00% |
| | Sepiplus ™ S | 0.80% |
| B | Proteol ™ OAT | 20.80% |
| | Oramix ™ NS 10 | 9.30% |
| | Amonyl ™ 265 BA | 5.10% |
| C | Composition (C_{1A}) | 2.00% |
| | Glyceryl glucoside | 1.00% |
| | Phenoxyethanol & ethylhexyl glycerol | 1.00% |
| | Perfume/Fragrance | 0.90% |
| | Colorant | 0.04% |

Procedure: disperse the Sepimax™ ZEN in the water and stir using a mechanical stirrer equipped with a deflocculator, a counter-rotating impeller and an anchor paddle, until a perfectly smooth gel is obtained. Add the Sepiplus™ S and then stir until the mixture is homogeneous. Next, add the ingredients of phase B, homogenize and individually add the additives of phase C. Adjust the pH to 6.0-6.5.

### C.10BB Cream

### Formula

| A | Easynov ™ | 2.30% |
|---|---|---|
| Lanol ™ 99 | | 1.00% |
| Sepimat ™ H10W | | 1.00% |
| Ethylhexyl methoxycinnamate | | 5.00% |
| B | Cyclomethicone | 6.00% |
| Triethoxycaprylsilane & alumina-silane & titanium oxide | | 8.00% |
| Red iron oxide & triethoxycaprylsilane | | 0.24% |
| Yellow iron oxide & triethoxycaprylsilane | | 0.66% |
| Black iron oxide & triethoxycaprylsilane | | 0.09% |
| Perfume/Fragrance | | 0.10% |
| C | Water qs | 100% |
| Sepinov ™ EMT10 | | 1.20% |
| D | Composition (C_{1A}) | 2.00% |

| | |
|---|---|
| Sepitonic ™ M3 | 1.00% |
| Phenoxyethanol & ethylhexyl glycerol | 1.00% |

Procedure: Prepare phase B by mixing the various ingredients and homogenize using a mixer equipped with a rotor-stator system at a spin speed of 4500 rpm, for a period of 6 minutes. Prepare phase C by adding the Sepinov™ EMT10 to the mixture of water and glycerol, and homogenize using a mixer equipped with a rotor-stator system at a spin speed of 4000 rpm for 4 minutes. Add phases A and B to phase C, and stir the resulting mixture using a mechanical stirrer equipped with an anchor paddle, at a speed of 30 rpm for 2 minutes, and then at a speed of 50 rpm for 20 minutes. Add the components of phase 5 one by one and stir at a speed of 50 rpm for 25 minutes.
C.11 High-Protection Antisun Spray with an SPF of Greater than 30

### Formula

| A | Montanov ™L | 1.00% |
|---|---|---|
| Montanov ™ 82 | | 1.00% |
| C12-15 Alkyl benzoate | | 17.00% |
| Dimethicone | | 3.00% |
| Octocrylene | | 6.00% |
| Ethylhexyl methoxycinnamate | | 6.00% |
| Bis(ethylhexyloxyphenol)methoxyphenyltriazine | | 3.00% |
| Tocopherol | | 0.05% |
| B | Water | qs 100% |
| C | Simulgel ™ INS 100 | 0.50% |
| Cyclodimethicone | | 5.00% |
| D | Composition (C_{1A}) | 3.00% |
| Phenoxyethanol & ethylhexyl glycerol | | 1.00% |
| Perfume/Fragrance | | 0.20% |
| E | Methylenebis(benzotriazolyl) | |

| | |
|---|---|
| Tetramethylbutylphenol | 10.00% |
| 25% Citric acid | qs pH = 5 |

Sepicalm™ S: Mixture of N-cocoyl amino acids, sarcosine, potassium aspartate and magnesium aspartate as described in WO 98/09611;
Proteol™ APL: Mixture of sodium salts of N-cocoyl amino acids, obtained by acylation of amino acids characteristic of apple juice;
Sepicide™ HB: Mixture of phenoxyethanol, methyl paraben, ethyl paraben, propyl paraben and butyl paraben, which is a preserving agent;
Capigel™ 98: Acrylate copolymer;
Sepicide™ CI: Imidazoline urea, which is a preserving agent;
Sepicide™ HB: Mixture of phenoxyethanol, methyl paraben, ethyl paraben, propyl paraben, butyl paraben and isobutyl paraben, which is a preserving agent;
Sepicalm™ VG: Mixture of N-palmitoyl proline in sodium salt form and of extract of Nymphea alba blossom;
Euxyl™ PE9010: Mixture of phenoxyethanol and ethylhexyl glycerol;
Proteol™ OAT: Mixture of N-lauryl amino acids obtained by total hydrolysis of oat protein as described in WO 94/26694;
Montaline™ C40: Chloride salt of monoethanolamine cocamidopropyl betainamide;
Amisoft™ CS-11: Sodium salt of N-cocoyl glutamate;
Simulsol™ 165: Mixture of PEG-100 stearate and glyceryl stearate;
Montanov™ 202 (arachidyl alcohol, behenyl alcohol and arachidyl glucoside) is a self-emulsifying composition such as those described in EP 0 977 626;
Lanol™99: Isononyl isononanoate;
Sepiplus™ 400: Self-invertible inverse latex of polyacrylates in polyisobutene and including polysorbate 20, as described in WO 2005/040230;
Lipacide™ C8G: Capryloylglycine sold by the company SEPPIC;
Micropearl™ M310: Crosslinked polymethyl methacrylate polymer in powder form, used as a texture modifier;
Sepimax™ Zen (INCI name: Polyacrylate Crosspolymer-6): Thickening polymer in the form of a powder;
Sepiplus™ S (INCI name: Hydroxyethyl Acrylate/Sodium Acryloyldimethyl Taurate Copolymer & Polyisobutene & PEG-7 Trimethylolpropane Coconut Ether): Self-invertible inverse latex;
Amonyl™ 265 BA (INCI name: cocoyl betaine): Foaming amphoteric surfactant;
Sepinov™ EMT10 (INCI name: Hydroxyethyl Acrylate/Sodium Acryloyldimethyl Taurate Copolymer): Thickening copolymer in the form of a powder;
Easynov™ (INCI name: Octyldodecanol and Octyldodecyl Xyloside and PEG-30 Dipolyhydroxystearate): Emulsifying agent of lipophilic tendency;
Sepimat™ H10 FW (INCI name: Methyl Methacrylate Crosspolymer and Squalane): Polymer used as texture agent;
Sepitonic™ M3 (INCI name: Magnesium Aspartate and Zinc Gluconate and Copper Gluconate): Mixture used as free-radical scavenger and energizing agent for cells;
Montanov™ L (INCI name: C14-22 Alcohols and C12-20 Alkylglucoside): Emulsifying agent;
Montanov™ 82 (INCI name: Cetearyl Alcohol and Coco-glucoside): Emulsifying agent;
Simulgel™ INS100 (INCI name: Hydroxyethyl Acrylate/Sodium Acryloyldimethyl Taurate Copolymer and isohexadecane and Polysorbate 60): Polymeric thickener.

1. A process for preparing a composition (C₁) comprising the following successive steps:
a) cultivation under soilless conditions of the plant Arctium lappa fed with a nutrient solution, so as to obtain a biomass (BM₁);
b) immersion of the roots of said biomass (BM₁) obtained in the preceding step a) in a medium (S₁), such that the biomass (BM₁)/mixture (S₁) ratio is between 0.5 kg/l and 1.5 kg/l, said medium (S₁) comprising, per 100% of its own mass, from 20% to 35% by mass of water, the pH of which has been adjusted to a value of between 1.5 and 3.5 by addition of a protic acid chosen from sulfuric acid, phosphoric acid and hydrochloric acid, and from 65% to 80% by mass of an organic solvent (OS₁) chosen from 1,2-propanediol, 1,3-propanediol, 1,4-butanediol, 1,3-butanediol, 1,2-butanediol, 2-methyl-2,4-pentanediol, 1,6-hexanediol, 1,8-octanediol or a mixture of these diols;
c) separation of the roots of the biomass on conclusion of the treatment defined in step b), in order to isolate a liquid phase (L₁);
d) immersion of the roots of said biomass (BM₂) resulting from step c) in said medium (S₁); in a biomass (BM₂)/mixture (S₁) ratio of between 0.1 kg/l and 1.5 kg/l;
e) separation of said biomass (BM₂) on conclusion of the treatment defined in step d), in order to isolate a liquid phase (L₂);
f) filtration of said liquid phase (L₃) obtained in step d), in order to isolate a liquid phase (L₃), and
g) mixing said liquid phases (L₁) and (L₃), then, if necessary, of addition of water and/or of said organic solvent (OS₁), so as to obtain the composition (C₁).

2. The process of claim 1 wherein the composition (C₁) comprises, per 100% of mass:
a)- from 60.0% by mass to 75.0% by mass of an organic solvent (OS₁) chosen from 1,2-propanediol, 1,3-propanediol, 1,4-butanediol, 1,3-butanediol, 1,2-butanediol, 2-methyl-2,4-pentanediol, 1,6-hexanediol, 1,8-octanediol, or a mixture of these compounds;
b)- from 0.1% by mass to 2.0% by mass of a composition (ES) comprising an amount by mass x₁, expressed as mass equivalent of 1-O-(2-caffeoyl)maloyl-3,5-di-O-caffeoylquinic acid, of greater than or equal to 200 mg/g of at least one compound of general formula (I): 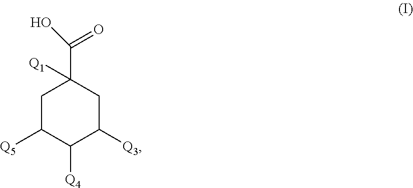
in which Q₁, Q₂, Q₃, Q₄ and Q₅ represent, independently of each other, a hydroxyl radical or a salt thereof or a radical chosen from:
(i)- the caffeoyl radical of formula (II): 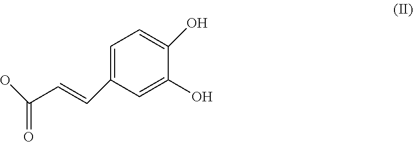
(ii)- the maloyl radical of formula (IIIa) or (Ib): 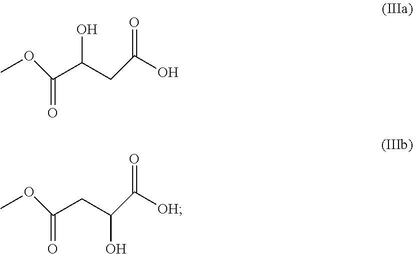
(iii)- the caffeoyl maloyl radical of formula (IVa) or (Ib): 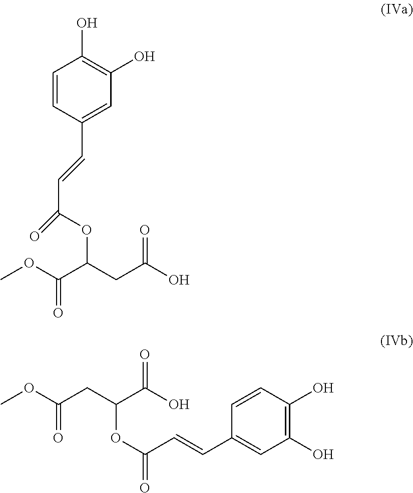
(iv)- the maloyl caffeoyl radical of formula (Va), (Vb), (Vc) or (Vd), 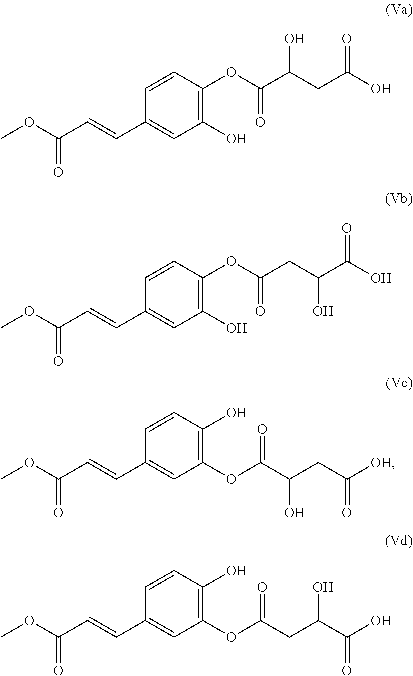
it being understood that at least one of these radicals Q₁, Q₂, Q₃, Q₄ and Q₅ represents neither the —OH radical nor a salt thereof; and
c)- from 20.0% by mass to 35.0% by mass of water.

3. The process of claim 2 wherein said composition (ES) comprises:
at least one compound of formula (Ia) corresponding to formula (I) for which Q₁ represents the maloyl radical of formula (IIIa) or of formula (IIIb) and Q₃ and Q₄ and Q₅, which are identical, each represent the caffeoyl radical of formula (II);
a compound of formula (Ib) corresponding to formula (I) for which Q₁ represents the caffeoylmaloyl radical of formula (IVa) or of formula (IVb), Q₃ and Q₅ each represent the caffeoyl radical of formula (II) and Q₄ represents a hydroxyl radical, and
at least one compound of formula (Ic) chosen from:
the compound of formula (I_{c1}) corresponding to formula (I) for which Q₁ and Q₅ each represent the caffeoyl radical of formula (II), Q₃ represents a hydroxyl radical and Q₄ represents the caffeoylmaloyl radical of formula (IVa) or of formula (IVb); and
the compound of formula (I_{c2}) corresponding to formula (I) for which Q₁ and Q₄ represent the caffeoyl radical of formula (II), Q₃ represents a hydroxyl radical and Q₅ represents the caffeoylmaloyl radical of formula (IVa) or of formula (IVb).

4. The process of claim 2 wherein the organic solvent (OS₁) is 1,2-propanediol.
